(19) United States
(12) Patent Application Publication
(10) Pub. No.: US 2005/0154276 A1
(43) Pub. Date: Jul. 14, 2005

(54) Apparatus and process for reading radiation reflected from human skin

(76) Inventors: Alessandro Barducci, Campi Bisenzio (IT); Roberto Casini, Prato (IT); Claudio De Felice, Monteriggioni (IT)

Correspondence Address: ARENT FOX PLLC, 1050 CONNECTICUT AVENUE, N.W., SUITE 400, WASHINGTON, DC, 20036, US

(21) Appl. No.: 10/508,543

(22) PCT Filed: Mar. 17, 2003
(86) PCT No.: PCT/IB03/01054

(30) Foreign Application Priority Data
Mar. 28, 2002 (IT) BO2002 A 000164

(51) Int. Cl. A61B 5/05

(52) U.S. Cl. 600/407; 600/476

(57) ABSTRACT

An apparatus for reading radiation from human skin and processing it for diagnostic purposes comprises reading means (10), for reading a main radiation (100) from an individual's skin, and a dispersive element (20), for separating the main radiation (100) into a plurality of portions (110) with different wavelengths. The apparatus (1) also has a transducer block (30), for receiving the portions (110) and generating a corresponding transmission signal (120), representative of the portions (110), and a processing block (50), which, according to the transmission signal (120), can calculate a predetermined number of physiological parameters characteristic of the individual's skin.

### TECHNICAL FIELD

The present invention relates to an apparatus for reading radiation from human skin and processing it for diagnostic purposes.

More specifically, the apparatus disclosed can evaluate the spectral reflectance and/or transmittance of an individual's skin, irrespective of their age, race and sex, providing at output useful indications for determining their health status.

### BACKGROUND ART

As is known, to detect the onset of various types of disorders, both clinical and pathological, reference may be made to some properties of the patient's skin, amongst which colour characteristics are of particular importance.

It has been scientifically demonstrated how the colour of the skin, in particular that of new-born babies, may be considered a general indicator, extremely sensitive to alterations in some physiological processes due to the state of the patient's health.

This particular property of human skin is due to the fact that, within its layers, the skin has a given number of pigments (technically called chromophores), such as free haemoglobin, saturated haemoglobin, melanin and bilirubin, whose concentration is heavily influenced by the patient's clinical conditions.

Therefore, it is obvious how, when particular pathologies occur, the shade of skin colour may vary, providing an important indication that the patient's health is worsening.

At present, the possibilities for taking precise, reliable readings of this type are very limited.

Very often, people depend on the experience of the doctor who, depending on the cases examined during his or her career, may be able to use exclusively his or her eyesight to recognise any changes in the colour of the patient's skin.

The limitations of this type of assessment are obvious.

Firstly, the reliability of the assessment and the subsequent diagnosis are based entirely on the doctor's abilities and experience, and however expert and competent the doctor is, he or she may still make mistakes in evaluations.

Moreover, it is important not to overlook all of the environmental aspects which can influence subjective perception, by the doctor performing the analysis, of the colour of the patient's skin, such as the ambient light, or the doctor's own psychophysical conditions.

Primary importance must also be given to the fact that, in the absence of extremely expert, qualified personnel, a colour analysis like the one described above cannot be performed.

There are also apparatuses available on the market which read light radiation from human skin. However, each of these apparatuses can only operate at a single, predetermined wavelength, and is designed to measure a single physiological parameter.

Therefore, these apparatuses provide information relative to only one precise chromophore (typically, the chromophore which is particularly reactive and sensitive at the wavelength considered).

Thus, the disadvantage of devices of the known type lies in the fact that they cannot provide a complete analysis of the concentration of the various pigments in the patient's skin, preventing a clear understanding of the patient's clinical conditions.

### DISCLOSURE OF THE INVENTION

The aim of the present invention is, therefore, to overcome the above-mentioned disadvantages.

In particular, the aim of the present invention is to provide an apparatus for reading radiation from human skin and processing it for diagnostic purposes, which can read the concentration of two or more pigments characteristic of the skin.

Another aim of the present invention is to provide an apparatus for reading radiation from human skin and processing it for diagnostic purposes, which constitutes a reliable non-invasive instrument for reading and analysing the patient's health status.

A further aim of the present invention is to provide an apparatus which can automatically process the physiological parameters read, to obtain a complete picture of the patient's health status.

Yet another aim of the present invention is to provide an apparatus for reading radiation from human skin and processing it for diagnostic purposes, which allows the data relative to each patient to be displayed and analysed even at a remote station, separated from the patient by a given distance.

The auxiliary aim of the present invention is to provide an apparatus for reading radiation from human skin and processing it for diagnostic purposes, which, once the analysis has been performed, allows the data collected to be archived electronically.

Still another aim of the present invention is to provide an apparatus for reading radiation from human skin and processing it for diagnostic purposes which is user friendly and can be used instantly, even by personnel without particular experience.

### BRIEF DESCRIPTION OF THE DRAWINGS

The technical features of the present invention, in accordance with the above-mentioned aims, are set out in the claims herein and the advantages more clearly illustrated in the detailed description which follows, with reference to the accompanying drawings, which illustrate a preferred embodiment, without limiting the scope of its application, and in which:

FIG. 1 is a block diagram of an apparatus according to the present invention;

FIG. 2 is a block diagram of a circuit element of the apparatus illustrated in FIG. 1.

### DETAILED DESCRIPTION OF THE PREFERRED EMBODIMENTS OF THE INVENTION

With reference to the accompanying drawings, the apparatus for reading radiation from human skin and processing it for diagnostic purposes is labelled 1 as a whole.

The apparatus 1 is designed to read a main radiation 100, from an individual's skin and, according to the processing of the main radiation 100—described in detail below—to automatically provide a diagnosis relative to the individual's health status.

Generally speaking, the main radiation 100 may be reflected, transmitted and/or backscattered by the skin of the individual examined. By way of example, in the case considered herein the main radiation 100 is reflected by the skin.

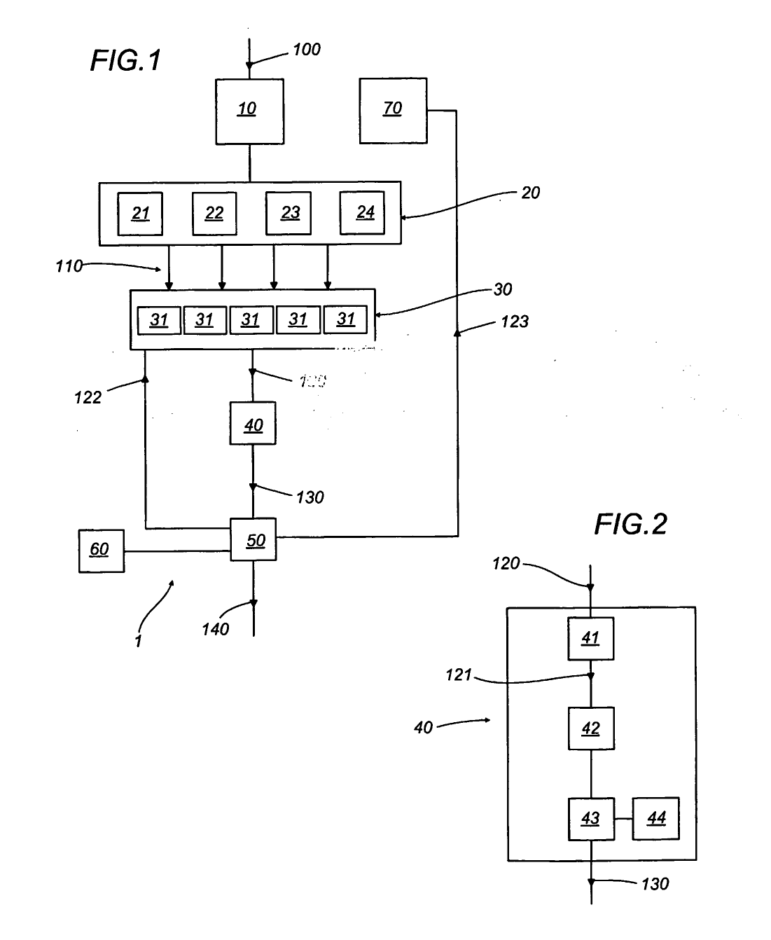

With particular reference to FIG. 1, the apparatus 1 comprises firstly reading means 10, for receiving the radiation 100 from the skin of the individual examined. The reading means 10 may be made in the form of a sensor, attached to a fibre optic channel.

Connected downstream of the reading means 10 there is a dispersive element 20, designed to separate the main radiation 100 into a plurality of portions 110, according to their wavelengths. Basically, the dispersive element 20 transmits the various portions 110 of the main radiation 100 in different trajectories or directions, according to the wavelength of each portion. This means that, for example, portions with a higher wavelength are transmitted at greater angles than portions with lower wavelengths.

In addition or as an alternative to the above, the division of the main radiation 100 into its portions 110 occurs by transmitting each portion 110 in a corresponding time interval, according to the wavelength of the portion. For example, portions with higher wavelengths can be transmitted with a longer time delay than portions with lower wavelengths.

To perform the above-mentioned operation, the dispersive element 20 may comprise diffraction means 21, refraction means 22, interference means 23 or filtering means 24.

In FIG. 1, the dispersive element 20 is illustrated as the combination of the various means 21, 22, 23 and 24. It is obvious how the dispersive element 20, according to requirements, can even comprise only one or more of the above-mentioned diffraction means 21, refraction means 22, interference means 23 and filtering means 24.

Each of the means 21, 22, 23 and 24 may be made using a lattice, and/or a prism, and/or an interferometer and/or a filter.

Moreover, the dispersive element 20 may be connected to one or more auxiliary dispersive elements, not illustrated, which can help to improve the quality of the functions performed by the dispersive element 20. In particular, the dispersive elements can be set up to carry out a kind of “optical pre-filtering” of the main radiation 100.

The portions 110 of the main radiation 100, from the dispersive element 20, are received by a transducer block 30 (sensor), which generates at output a transmission signal 120, representative of the various portions 110. In particular, since the optical main radiation (the main radiation 100 normally has components whose wavelength lies within the range of the visible and/or close, medium and thermal infrared), the transducer block 30 generates a transmission signal 120 which is electrical, to send the information relative to the above-mentioned portions 110 to the circuitry connected downstream.

In a preferred embodiment, the transducer block 30 comprises a predetermined number of light-sensitive elements 31, designed to receive at least one of the portions 110 of the main radiation 100 and to generate the transmission signal 120. Advantageously, the latter may be a function of the power of the portions 110 and, in particular, proportional to the power of the portions 110.

Depending on requirements, the light-sensitive elements 31 may be of the Charge Coupled Device (CCD) type, or bolometers, or arrays of photodiodes, or sensors with CMOS direct addressing (non-sequential shift register) technology.

The apparatus 1 also has an acquisition interface 40, connected downstream of the light-sensitive elements 31. The acquisition interface 40 is designed to receive the above-mentioned transmission signal 120 and to provide at output a corresponding digital auxiliary signal 130, representative of the optical properties of the skin of the individual examined. In particular, the digital auxiliary signal 130 may incorporate the irradiance reflected and/or transmitted by the skin. The latter measurement, as is known to experts in the field, indicates the skin's absorption—reflection characteristics relative to incident radiation.

More specifically, the acquisition interface 40 comprises an amplifier and compensator block 41, designed to receive the transmission signal 120, amplify it and compensate some unwanted effects (for example, offset conditions which are not ideal). Therefore, the block 41 supplies at output an amplified, compensated signal 121.

The amplified, compensated signal 121 is received by an analogue-to-digital converter 42, which converts it to digital form.

It is important to emphasise how, by means of the block 41 and converter 42, the transmission signal 120 undergoes only “formal” processing, leaving the information it contains substantially unchanged. So, obviously the signal at the analogue-to-digital converter 42 output may incorporate the same information as is contained in the transmission signal 120.

The acquisition interface 40 also has a main memory 44, for temporarily or permanently storing the information contained in the transmission signal 120. The main memory 44 may be connected to a microcontroller 43, designed to manage the entire operation of the acquisition interface 40. The microcontroller in question may even be the CPU of a control computer.

Specifically, the microcontroller 43 is designed to receive the signal supplied at output by the converter 42 and to save the content in the main memory 44. To allow further processing of this information, the microcontroller 43 is also designed to generate at output the above-mentioned digital auxiliary signal 130, incorporating in it all data which must be transmitted to the downstream circuitry.

In addition to the above, the microcontroller 43 may be given the task of generating a scan signal 122, to activate the transducer block 30. Otherwise the scan signal(s) may be generated by dedicated electronic circuitry made, for example, using programmable logics.

Considering, by way of example, the case in which the transducer block 30 is obtained with a CCD matrix, the microcontroller generates the scan signal 122 to activate collection of the charge accumulated in each device and the consequent transfer of these charges in the transmission signal 120.

Connected downstream of the acquisition interface 40, and in particular of the microcontroller 43, there is a processing block 50, which, according to the information collected by the elements described above, calculates a predetermined number of physiological parameters of the skin of the individual under observation.

More specifically, the processing block 50 performs all operations necessary to obtain a plurality of physiological parameters of the individual's skin.

In further detail, the processing block 50 is firstly designed to receive the digital auxiliary signal 130 from the microcontroller 43. The processing block 50 uses the auxiliary signal 130 to calculate a function characteristic of the individual's skin. The characteristic function is, preferably, the spectral reflectance or transmittance, which represents the skin's absorption or reflection properties according to the wavelength.

The spectral reflectance or transmittance may be calculated as the ratio of the incident irradiance (that is to say, the intensity of an incident radiation, considered at various wavelengths) to the reflected/transmitted irradiance (that is to say, the intensity of the reflected/transmitted radiation, considered at different wavelengths).

To calculate the above-mentioned physiological parameters, the processing block 50 compares the characteristic function, obtained as indicated above, with a function which was saved in the memory previously. The latter basically consists of a predetermined mathematical function, in which the wavelength and the physiological parameters relative to the individual's skin are left as free parameters. Therefore, comparing the characteristic function, obtained by means of experimental readings, with the presaved function allows the values to be set for the physiological parameters present in the presaved function.

In further detail, the comparison between the characteristic function and the presaved function may consist in the generation of a cost function, which depends, for example, on the difference between the two functions, and the subsequent application of an optimisation algorithm, to obtain the desired results.

A cost function which may be used by the apparatus 1 is as follows:

$${χ}^{2} = \int \frac{{\vert ρ ( λ ) - R ( λ ) \vert}^{2}}{{err}^{2} ( λ )} ⅆ λ$$

where:
the total is calculated relative to a range of wavelengths covering the entire visible spectrum;
x² is the cost function;
ρ(λ) is the presaved function;
R(λ) is the characteristic function;
err²(λ) indicates the error in the individual measurement of the skin's reflectance/transmittance spectrum.

In this particular case, the optimisation algorithm may be a minimisation algorithm which, by minimising the above-mentioned cost function, allows minimisation of the difference between the characteristic function and the presaved function according to the latter's free parameters (that is to say, the relevant physiological parameters), giving the skin's characteristic physiological parameters.

In other words, using a suitable mathematical model and making it adhere as far as possible to the experimental data collected, the values relative to the relevant physiological parameters are calculated.

In a preferred embodiment, the physiological parameters may be selected in such a way that they are a function of the concentrations of predetermined chromophores in the skin of the individual under observation. In particular, each physiological parameter may be a function of the concentration of a respective chromophore.

Suitably, each physiological parameter is proportional to the concentration of the corresponding chromophore and, preferably, each physiological parameter coincides with the concentration of the chromophore associated with it. For example, the chromophores whose concentration may be interesting are: free haemoglobin, saturated haemoglobin, melanin, bilirubin and carotenoids.

In this case, the apparatus 1, by means of the elements described above, can receive the main radiation 100, from the skin of the individual examined, and calculate the spectral reflectance, based on the readings taken. The spectral reflectance obtained in this way is then compared with the presaved function, calculating the concentrations of the skin's various chromophores which, as indicated above, may be suitably interpreted to build up a clinical picture of the patient's condition.

In practice, it is important to consider than the trend, according to the wavelength, of the spectral irradiance of an individual's skin is determined by the overlapping of the effects due to the various chromophores present, according to the concentration.

This means that each chromophore has a characteristic trend, in terms of spectral irradiance, and more or less influences the form of the skin's overall spectral irradiance according to its concentration. A chromophore present in a high concentration will have a significant effect on the trend of the total spectral irradiance, whilst a chromophore present in a low concentration will have a lesser effect.

The problem solved here is that of identifying the concentrations of the various pigments, knowing only the total spectral irradiance (obtained from the main radiation 100 reading and subsequent processing of its portions 110) and the characteristic trend of the spectral irradiance of each chromophore.

A mathematical model (the presaved function) is created which is a function of the sum of the trends of each pigment, each of the trends being “weighed” (that is to say, multiplied by a suitable coefficient) by the concentration of the respective pigment. Obviously, the pigment concentrations are initially free parameters, whose values are set at a later stage.

To calculate the values of the physiological parameters, the cost function is generated, which advantageously depends on the difference between the spectral irradiance detected and the predetermined mathematical model. Finally, the minimisation algorithm is applied and the mathematical model made to correspond as far as possible to the experimental data collected (that is to say, the spectral irradiance). In other words, the pigment concentration values at which the difference between the characteristic function and the presaved function is at a minimum are calculated.

In order to save the above-mentioned presaved function, the processing block 50 is connected to an auxiliary memory 60. The latter is also designed to contain a predetermined number of reference parameters, whose use is described below.

Once the physiological parameters have been obtained, to complete the processing for which it is designed and supply an automatic diagnosis at output, the processing block 50 compares the physiological parameters and the reference parameters contained in the auxiliary memory 60, and, as a result of this comparison, generates an output signal 140, representative of the health of the individual examined.

More specifically, the reference parameters are divided, within the auxiliary memory 60, into a plurality of groups, each associated with a respective clinical picture. In practice, there is a plurality of clinical pictures which may be assigned to the individual under observation. Each clinical picture is assigned a group of reference parameters which, following studies on the subject, were considered representative of such a clinical picture.

Therefore, by comparing the physiological parameters obtained by means of experimental readings with the presaved reference parameters, the set of reference parameters closest to the physiological parameters can be selected, so that the processing block 50 can incorporate the clinical picture associated with the selected group of reference parameters in the output signal 140.

Advantageously, the apparatus 1 may also be equipped with an actuator 70, preferably mechanical, attached to the reading means 10 and designed to cover them, preventing them from reading the main radiation 100.

In practice, the actuator 70 may have a covering element which, depending on a suitable activation signal 123 generated by the microcontroller 43, is positioned at a reading input on the reading means 10, so that the reading means 10 can no longer receive the main radiation 100 or any of the other light radiation. In this way, the apparatus 1 can measure the so-called “dark signal”, that is to say, it can operate for a predetermined period of time without radiation at input and so can update its operating parameters, with which the signals subsequently read will be compared.

Moreover, it is important to emphasise how the processing block 50 can be made like a conventional PC and may be connected to the acquisition interface 40 microcontroller 43, for example, by a serial line, for example of the RS232 type.

The processing block 50 may also be connected to other similar devices, for example by means of the Internet, so that the data read by the apparatus 1 can also be examined by a doctor who is not on the spot, but who has a remote computerised workstation at his or her disposal.

Another function which may be performed by the processing block 50 is the generation of alarm signals, to activate suitable indication devices, designed to notify personnel when the values read relative to a patient examined have exceeded a predetermined caution threshold.

Moreover, by means of the auxiliary memory 60, and, if present, storage devices connected to other computers linked to the processing block 50, an archive can be created, designed to hold the data relative to many individuals, and many tests to which each individual was subjected, so that a vast database is available, useful at clinical, scientific and legal levels.

The following is a description of the process used by the apparatus 1 to obtain the parameters described above.

Firstly, the main electromagnetic radiation 100 from the skin of the individual examined is read. This main radiation 100 may, generally speaking, be reflected, transmitted and/or backscattered by the skin.

Then, the main radiation 100 is separated into at least two portions 110, with different wavelengths. This separation step can be performed by taking advantage of physical effects, such as diffraction, refraction, diffusion, interference or filtering.

In particular, each portion 110 may be transmitted in a different trajectory, according to its wavelength, or in a different time interval to the other portions 110, with different wavelengths.

It is important to emphasise how the two types of separation indicated here—spatial and temporal separation—may be used either alternatively, or in combination with one another.

Therefore, a transmission signal 120 is generated according to the various portions 110. These are light portions and, in order to process the information contained in them, they must be converted into the transmission signal 120, which is electrical.

In more detail, the transmission signal 120 may be a function of the electromagnetic power of the portions 110 and, more specifically, proportional to the power of the portions 110.

The transmission signal 120, initially analogue, is converted to digital form, and the information incorporated in it is saved, preferably in the afore-mentioned main memory 44.

This information is then processed to obtain the characteristic function of the individual's skin, that is to say, its spectral reflectance. As indicated above, the spectral reflectance is then compared with the presaved function, that is to say, a mathematical model in which the relevant physiological parameters are left as free parameters, and following this comparison said physiological parameters are calculated.

The comparison between spectral irradiance and the presaved function is performed by generating a cost function, preferably with the form indicated above. The cost function depends on the difference between the spectral irradiance read and the presaved function and, by applying an optimisation algorithm—in particular, a minimisation algorithm—to the cost function, the physiological parameters characteristic of the skin of the person under observation can be obtained.

As indicated above, the physiological parameters calculated in this way may be a function of the concentration of the chromophores present in the patient's skin and, preferably, may be proportional to these concentrations.

In a preferred embodiment, the physiological parameters coincide with the concentrations of the relevant chromophores in the patient's skin. In other words, each physiological parameter represents the concentration of a corresponding chromophore in the skin of the person under observation.

Once the values of the above-mentioned physiological parameters have been calculated, they are compared with a predetermined number of presaved reference parameters.

The reference parameters are advantageously divided into a plurality of groups. Each group is associated with a respective clinical picture. In practice, there is a plurality of clinical pictures which may be assigned to the individual under observation. Therefore, each clinical picture is associated with a group of reference parameters which, following studies carried out on the subject, were considered representative of said clinical picture.

Thus, a comparison of the physiological parameters obtained by means of experimental readings and the presaved reference parameters allows the selection of a set of reference parameters closest to the physiological parameters, and the supply of a suitable output signal 140, incorporating the clinical picture associated with the selected group of reference parameters.

The invention has important advantages.

Firstly, the apparatus and process disclosed allow a measurement and the relative diagnosis to be made in a non-invasive way, that is to say, by simply applying a sensor to the patient's skin, the diagnosis also being extremely precise and reliable.

Use of the invention may sometimes replace more complex, invasive and more expensive tests (for example, blood tests), thus also simplifying and cutting the cost of medical and health care.

Moreover, all of the operations necessary to obtain the desired results are performed substantially automatically, meaning that even non-specialised personnel can perform the procedure.

Another advantage of the present invention is the fact that the automatic diagnosis ultimately provided is extremely complete and accurate, since it is based on the reading and on the analysis of a plurality of patient physiological parameters and, in particular, on the reading and analysis of the concentration of a plurality of pigments in the skin of the individual under observation.

Finally, a further advantage of the present invention is highlighted by the fact that the data collected, converted to digital form and processed, can be archived, for scientific, clinical and legal purposes, and can be transmitted using telecommunications networks (for example, the Internet), so that the health of the person examined can even be evaluated from remote computerised workstations.

The invention described can be subject to numerous modifications and variations without thereby departing from the scope of the inventive concept. Moreover, all the details of the invention may be substituted by technically equivalent elements.

1. An apparatus for reading radiation from human skin and processing it for diagnostic purposes, comprising: reading means (10), for reading a main electromagnetic radiation (100) from an individual's skin; at least one dispersive element (20), being connected downstream of the reading means (10) to separate the main radiation (100) into at least two portions (110) with different wavelengths; a transducer block (30), designed to receive the portions (110) of the main radiation (100) and to generate at output a corresponding transmission signal (120), representing the portions (110); a processing block (50), being connected downstream of the transducer block (30) and designed to calculate a predetermined number of physiological parameters characteristic of the individual's skin, according to the transmission signal (120): characterised in that it further comprises an actuator (70) attached to the reading means (10) and designed to cover the reading means (10), preventing them from reading the main radiation (100) from the individual's skin.

2. The apparatus according to claim 1, characterised in that the processing block (50) is designed to calculate a plurality of physiological parameters characteristic of the individual's skin, according to the transmission signal (120).

3. The apparatus according to claim 1, characterised in that the dispersive element (20) comprises diffraction means (21) to transmit each of the portions (110) of the main radiation (100) in a corresponding trajectory and/or in a corresponding time interval, according to the wavelength of each of the portions (110), the diffraction means (21) preferably having a lattice and/or a prism and/or an interferometer and/or a filter.

4. The apparatus according to claim 1, characterised in that the dispersive element (20) comprises refraction means (22) to transmit each of the portions (110) of the main radiation (100) in a corresponding trajectory and/or in a corresponding time interval, according to the wavelength of each of the portions (110), the refraction means (22) preferably having a lattice and/or a prism and/or an interferometer and/or a filter.

5. The apparatus according to claim 1, characterised in that the dispersive element (20) comprises interference means (23) to transmit each of the portions (110) of the main radiation (100) in a corresponding trajectory and/or in a corresponding time interval, according to the wavelength of each of the portions (110), the interference means (23) preferably having a lattice and/or a prism and/or an interferometer and/or a filter.

6. The apparatus according to claim 1, characterised in that the dispersive element (20) comprises filter means (24) to transmit each of the portions (110) of the main radiation (100) in a corresponding trajectory and/or in a corresponding time interval, according to the wavelength of each of the portions (110), the filter means (24) preferably having a lattice and/or a prism and/or an interferometer and/or a filter.

7. The apparatus according to claim 1, characterised in that the transducer block (30) comprises a predetermined number of light-sensitive elements (31), selected from one of Charge Coupled Device (CCD) type, or bolometers, or arrays of photodiodes, or sensors with CMOS direct addressing (non-sequential shift register) technology, being able to receive at input at least one predetermined portion (110) of the optical main radiation (100) and to supply at output the transmission signal (120), the signal being electrical and, being a function of the power of the portions (110).

8. The apparatus according to claim 1 or 7, characterised in that it also comprises an acquisition interface (40), between the light-sensitive elements (31) and the processing block (50), the acquisition interface (40) being designed to receive the transmission signal (120) and, according to the transmission signal (120), to generate at output a corresponding digital auxiliary signal (130), representative of optical properties of the individual's skin and, in particular, of a reflected and/or transmitted irradiance of the individual's skin.

9. The apparatus according to claim 8, characterised in that the acquisition interface (40) comprises: an amplifier and compensator block (41), for receiving the transmission signal (120) and supplying an amplified and compensated signal (121) at output; an analogue-to-digital converter (42), for converting the amplified and compensated signal (121) from the amplifier and compensator block (41) to digital form; a main memory (44), for saving at least the information incorporated in the transmission signal (120); a microcontroller (43), connected to the analogue-to-digital converter (42), to the main memory (44) and to the transducer block (30) and being designed to:
generate a scan signal (122), for activating the transducer block (30);
receive the digital signal from the analogue-to-digital converter (42) output;
save the information incorporated in the transmission signal (120) in the main memory (44);
supply at the processing block (50) output the information saved in the main memory (44), incorporating it in the digital auxiliary signal (130).

10. The apparatus according to claim 9 characterised in that the actuator (70), is preferably a mechanical actuator, the microcontroller (43) also being able to generate an activation signal (123) for the mechanical actuator (70).

11. The apparatus according to claim 8 characterised in that the processing block (50) is designed to:
receive the auxiliary signal (130);
process the information incorporated in the auxiliary signal (130), to obtain a function characteristic of the individual's skin, said characteristic function preferably being a spectral reflectance and/or transmittance of the individual's skin;
compare the characteristic function with a presaved function, to obtain the physiological parameters characteristic of the individual's skin, according to the comparison.

12. The apparatus according to claim 11 characterised in that the processing block (50) is also designed to generate a cost function, according to the comparison between the characteristic function and the presaved function, and to apply an optimisation algorithm, in particular a minimisation algorithm, to the cost function, to obtain the physiological parameters.

13. The apparatus according to claim 1, characterised in that it also comprises an auxiliary memory (60), connected to the processing block (50), for saving the presaved function and a predetermined number of reference parameters, the processing block (50) also being designed to:
compare the physiological parameters with the reference parameters;
according to the comparison, generate an output signal (140), representative of the individual's health status.

14. The apparatus according to claim 1, characterised in that each of the physiological parameters is a function of the concentration of a corresponding chromophore in the patient's skin, each of the physiological parameters preferably being proportional and, in particular, substantially equal to the concentration of said corresponding chromophore.

15. A process for reading radiation from human skin and processing it for diagnostic purposes, characterised in that it comprises the following steps:
reading a main electromagnetic radiation (100), from an individual's skin;
separating the main radiation (100) into at least two portions (110), having different wavelengths;
according to the portions (110) of the main radiation (100), calculating a predetermined number of physiological parameters characteristic of the individual's skin.

16. The process according to claim 15, characterised in that the calculation step comprises an auxiliary calculation sub-step, for calculating a plurality of physiological parameters characteristic of the individual's skin, according to the portions (110) of the main radiation (100).

17. The process according to claim 15 or 16, characterised in that it also comprises a step of transmitting each of the portions (110) of the main radiation (100) in a corresponding trajectory and/or in a corresponding time interval, according to the wavelength of each of the portions (110), the transmission step preferably following the separation step.

18. The process according to claim 17, characterised in that it also comprises, according to the transmission step, a step for generation of a transmission signal (120), which is a function of the power of the portions (110) of the main radiation (100) and, in particular, proportional to the power of the portions (110).

19. The process according to claim 18, characterised in that is also comprises the following steps:
converting the transmission signal (120) into digital form;
saving the information incorporated in the transmission signal (120).

20. The process according to claim 15, characterised in that it also comprises a transduction step, in which the portions (110) of the main, optical radiation (100) are converted into the transmission signal (120), the latter preferably being electrical.

21. The process according to claim 15, characterised in that it also comprises the following steps:
processing of the information incorporated in the transmission signal (120), to obtain a function characteristic of the individual's skin, said characteristic function preferably being a spectral reflectance and/or transmittance of the individual's skin;
comparing the characteristic function with a presaved function, to obtain the physiological parameters characteristic of the individual's skin, according to the comparison.

22. The process according to claim 21, characterised in that the processing step comprises the following sub-steps:
generating a cost function, according to the comparison between the characteristic function and the presaved function;
applying an optimisation algorithm, in particular a minimisation algorithm, to the cost function in order to obtain the physiological parameters.

23. The process according to one of claims 21 or 22, characterised in that it also comprises the following steps, preferably after the processing step:
comparing the physiological parameters with a predetermined number of reference parameters;
generating an output signal (140), representative of the individual's health status, according to the comparison.

24. The process according to claim 21, characterised in that each of the physiological parameters is a function of the concentration of a corresponding chromophore in the patient's skin, each of the physiological parameters preferably being proportional and, in particular, equal to the concentration of the corresponding chromophore.
